(19) United States
(12) Patent Application Publication
(10) Pub. No.: US 2025/0288555 A1
(43) Pub. Date: Sep. 18, 2025

(54) SYSTEMS AND METHODS TO IMPROVE EXERCISE TOLERANCE

(71) Applicant: EpicentRx, Inc., La Jolla, CA (US)

(72) Inventors: Bryan ORONSKY, Los Altos Hills, CA (US); Scott CAROEN, San Francisco, CA (US); Tony R. REID, San Diego, CA (US)

(73) Assignee: EpicentRx, Inc., La Jolla, CA (US)

(21) Appl. No.: 18/861,496

(22) PCT Filed: May 1, 2023
(86) PCT No.: PCT/US2023/020563

§ 371 Date: Oct. 29, 2024

Related U.S. Application Data
(60) Provisional application No. 63/337,543, filed on May 2, 2022.

(51) Int. Cl. A61K 31/397; A61K 33/26; A61K 35/14; A61K 35/18; A61K 45/06; A61P 21/00

(52) U.S. Cl. CPC A61K 31/397; A61K 33/26; A61K 35/14; A61K 35/18; A61K 45/06; A61P 21/00

(57) ABSTRACT

Methods and combinations for enhancing physical performance of a mammal are provided. A method includes administering an effective amount (e.g., therapeutically effective amount) of RRx-001, or a pharmaceutically acceptable salt thereof, to said mammal prior to said physical performance. In some embodiments, the method further comprises administering an agent and/or a blood product before, during, or after the administration of the effective amount of RRx-001, or the pharmaceutically acceptable salt thereof.

### CROSS-REFERENCE TO RELATED APPLICATIONS

This application claims the benefit of and priority to U.S. Provisional Patent Application Ser. No. 63/337,543 filed on May 2, 2022, the contents of which are hereby incorporated by reference in their entirety.

### FIELD OF THE INVENTION

This disclosure relates to compositions and treatments to improve various aspects of physical performance. Specifically, this disclosure provides methods and compositions for enhancing physical performance in a subject in need thereof.

### BACKGROUND

It is well documented that poor aerobic exercise capacity directly correlates with morbidity, mortality, and quality of life. Physical inactivity/sedentary behavior initiates a vicious cycle of poor quality of life, deconditioning, sleep problems, weight gain, psychological problems, musculoskeletal pain, and metabolic disturbances that aggravate many disease symptoms and predispose to more physical inactivity/sedentary behavior. Poor exercise capacity is associated with a host of causes and conditions including heart and lung disease, general deconditioning and even some medications like statins, the side effects of which may include myalgia, creatine kinase (CK) elevations and rhabdomyolysis. So myriad and so irrefutable are the benefits of exercise on health and well-being that it is often described and prescribed as medicine. See Swisher A K. Yes, “Exercise is Medicine” . . , but It Is So Much More!. Cardiopulm Phys Ther J. 2010; 21(4):4.

A veritable “cottage industry” has sprung up around the many drugs, exercise-mimetics, nutritional supplements, herbal remedies, botanicals, and general lifestyle strategies with potential energy-boosting properties. However, a comprehensive, one-size-fits-all solution, which addresses how to improve exercise capacity and performance and health status is currently unknown. The fact that obesity has reached epidemic proportions in the United States and much of the Westernized world illustrates the unmet need for a better method to improve exercise capacity.

Therefore, a need exists for compositions that can improve various aspects of physical performance in the population post-exercise.

### SUMMARY

An embodiment of the disclosed subject matter is a method for enhancing physical performance, exercise tolerance and endurance time of a mammal prior to said physical performance by administration to said mammal prior to said physical performance of an effective amount (e.g., therapeutically effective amount) of RRx-001 in a range between about 0.5 mg-200 mg within 24 h of said physical performance. RRx-001, or a pharmaceutically acceptable salt thereof, may be administered once per day, once per week, twice per week, once per month, once every 3 months, or combinations thereof. The mammal may be a human. The human may be an athlete, a body builder, a manual worker, or an individual desiring to lose weight or enhance physique. The mammal may be non-human. The mammal may be a domesticated animal. The mammal may be a non-domesticated animal. The mammal may be a livestock animal. The mammal may be a horse. The mammal may be a dog.

Administering the effective amount of RRx-001, or the pharmaceutically acceptable salt thereof, occurs via oral administration, transdermal administration, administration by inhalation, nasal administration, topical administration, intra-aural administration, rectal administration, intravenous administration, intramuscular administration, subcutaneous administration, or intraperitoneal administration, or combinations thereof. In some embodiments, administering the effective amount of RRx-001, or the pharmaceutically acceptable salt thereof, occurs via parenteral administration. In some embodiments, administering the therapeutically effective amount of RRx-001, or the pharmaceutically acceptable salt thereof, is performed via a single administration.

In some embodiments, administering the therapeutically effective amount of RRx-001, or the pharmaceutically acceptable salt thereof, is performed via at least two administrations. In some embodiments, administering the therapeutically effective amount of RRx-001, or the pharmaceutically acceptable salt thereof, is performed at a frequency of about once per day. In some embodiments, administering the therapeutically effective amount of RRx-001, or the pharmaceutically acceptable salt thereof, is performed at a frequency of about once per week. In some embodiments, administering the therapeutically effective amount of RRx-001, or the pharmaceutically acceptable salt thereof, is performed at a frequency of about twice per week. In some embodiments, administering the therapeutically effective amount of RRx-001, or the pharmaceutically acceptable salt thereof, is performed at a frequency of about once per month. In some embodiments, administering the therapeutically effective amount of RRx-001, or the pharmaceutically acceptable salt thereof, is performed at a frequency of about once every three months.

The effective amount (e.g., therapeutically effective amount) of RRx-001 may result in at least one of increased VO₂ max, improved left ventricular (LV) function, reduced low-density lipoprotein (LDL), increased high-density lipoprotein (HDL) levels, decreased serum triglyceride (TAG) levels, improved fasting glycemia, improved insulin resistance, decreased blood pressure, delayed onset muscle soreness, delayed pain threshold, decreased slow skeletal troponin I concentration (sTnI), decreased serum myoglobin concentration, decreased serum IL-1β, IL-6, decreased 8-hydroxy-2′-deoxyguanosine (8-OHdG), decreased TNF-a concentration, reduced sedimentation rate and C-reactive protein, enhanced fatty acid metabolism, enhanced fatty acid oxidation, enhanced fatty acid utilization, or increased long-chain fatty acid (LCFA) muscle uptake. In some embodiments, peripheral vasodilation occurs in the subject subsequent to administration of the RRx-001, or a pharmaceutically acceptable salt thereof.

The mammal may be healthy. The mammal may have or may be suspected of having hypercholesterolemia, hyperlipidemia, cancer, muscular dystrophies, peripheral vascular disease, patent foramen ovale, obesity, type 2 diabetes, angina pectoris; heart failure, mitochondrial disorders or diseases, chronic obstructive pulmonary disease, hyperCKemia, neurodegenerative disease, motor neuron disease, neuromuscular disease, multiple sclerosis, Charcot-Marie-Tooth disease, myositis including polymyositis and dermatomyositis, cardiovascular disease, pulmonary artery hypertension, insulin resistance, hypertension, myoedema, rhabdomyolysis, idiopathic chronic muscle fatigue, reduced skeletal muscle function, disrupted skeletal muscle function or metabolism, cardiac abnormalities, or dysfunctional muscle, dysfunctional heart, and/or dysfunctional skeletal metabolism.

The method may further include administering an agent before, during, or after the administration of the effective amount of RRx-001, or the pharmaceutically acceptable salt thereof. In some embodiments, the agent is selected from the group consisting of: an antioxidant, a vitamin, a mineral, a corticosteroid, a human growth hormone, a steroid, levothyroxine, erythropoietin, a selective androgen receptor modulator (SARM), human chorionic gonadotrophin (HCG), adrenocorticotrophin (ACTH), insulin, a beta 2 agonist, marijuana, a diuretic, a narcotic, a beta blocker, a nitrite, a nitrate, a nitric oxide donor, a PDE5 inhibitor, a sympathomimetic drug, and an amino acid.

In some embodiments, the agent may comprise the antioxidant. The antioxidant may be selected from the group consisting of: lycopene, coenzyme Q10, melatonin, selenium, alpha-lipoic acid, ellagic acid, lutein, resveratrol, anthocyanins, ellagitannins, polyphenols, quercetin, curcumin. In some embodiments, the agent may comprise the vitamin. The vitamin may be selected from the group consisting of: Vitamin A. Vitamin C, Vitamin E, folic acid, and biopterin. The administering may include one or more minerals. In some embodiments, the agent may comprise the mineral, which may be selected from the group consisting of: calcium, phosphorus, potassium, sodium, chloride, magnesium, iron, zinc, iodine, chromium, copper, fluoride, molybdenum, and manganese.

In some embodiments, the agent may comprise a corticosteroid. In some embodiments, the agent may comprise human growth hormone. In some embodiments, the agent may comprise an anabolic steroid. In some embodiments, the agent may comprise levothyroxine. In some embodiments, the agent may comprise erythropoietin. In some embodiments, the agent may comprise a selective androgen receptor modulator (SARM). In some embodiments, the agent may comprise human chorionic gonadotrophin (HCG). In some embodiments, the agent may comprise adrenocorticotrophin (ACTH). In some embodiments, the agent may comprise insulin. In some embodiments, the agent may comprise a beta 2 agonist. In some embodiments, the agent may comprise marijuana. In some embodiments, the agent may comprise a diuretic. In some embodiments, the agent may comprise blood doping. In some embodiments, the agent may comprise a narcotic. In some embodiments, the agent may comprise a beta blocker.

In some embodiments, the agent may comprise a nitrite. In some embodiments, the agent may comprise a nitrate. In some embodiments, the agent may comprise a nitric oxide donor. The nitric oxide donor may include organic nitrates. The organic nitrates may be selected from the group consisting of: glyceryl trinitrate and isosorbide dinitrate. The nitric oxide donor may include sodium nitroprusside. The nitric oxide donor may include sydnonimines. The sydnonimines may be selected from the group consisting of: molsidomine and SIN-1. The nitric oxide donor may include S-nitrosothiols. The S-nitrosothiols may be selected from the group consisting of: s-nitrosoglutathione and SNAP. The nitric oxide donor may include NONOates. The NONOates may be selected from the group consisting of: spermine NONOate and DETA-NONOate. The administering may include a PDE5 inhibitor. The PDE5 inhibitor may be selected from the group consisting of: sildenafil (Viagra), tadalafil (Cialis), vardenafil (Levitra), and avanafil (Stendra). In some embodiments, the agent may comprise a sympathomimetic drug. The sympathomimetic drug may be selected from the group consisting of: dobutamine, albuterol, phenylpropanolamine, amphetamine, and ephedrine. In some embodiments, the agent may comprise one or more amino acids. The one or more amino acids may be selected from the group consisting of: L-camitine, L-creatine, L-taurine, arginine, and lysine.

In some embodiments, the therapeutically effective amount of the RRx-001, or the pharmaceutically acceptable salt thereof, is administered as a composition comprising a blood product. In some embodiments, the blood product comprises erythrocyte cells. In some embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In some embodiments, the blood product is a mixture of packed red blood cells. In some embodiments, the blood product is whole blood. In some embodiments, the whole blood is autologous whole blood or donor-matched allogenic whole blood.

In some embodiments, administering the effective amount of RRx-001, or the pharmaceutically acceptable salt thereof, occurs via oral administration. The administering may be in a form of an oral rinse. The administering may be in the form of a drink. The administering may be in the form of an oral pill or tablet. In other embodiments, the administering may be in the form of an intravenous infusion. In some embodiments, the administering may be in the form of a subcutaneous injection. In some embodiments, the administering may be through inhalation. In some embodiments, the administering may be sublingual. In some embodiments, the administering may be rectal.

Another embodiment describes a composition for enhancing physical performance in a subject, the composition comprising an effective amount of RRx-001, or a pharmaceutically acceptable salt thereof, and at least one pharmaceutically acceptable carrier or excipient. The composition includes the agent and/or the blood product.

### BRIEF DESCRIPTION OF THE DRAWINGS

FIG. 1 is a schematic of the study design to test exercise capacity in mice subsequent to administration of RRx-001.

FIG. 2 is a series of bar graphs that compare exercise mice treated with RRx-001 and control for three consecutive weeks.

FIG. 3 is a bar graph comparing levels of creatine kinase-MB found in mice after exercise.

FIG. 4A is a series of bar graphs comparing concentrations of malondialdehyde (MDA) in gastrocnemius, soleus, and extensor digitorum longus muscles.

FIG. 4B is a series of bar graphs comparing concentrations of superoxide dismutase (SOD) in gastrocnemius, soleus, and extensor digitorum longus muscles.

FIG. 4C is a series of bar graphs comparing diameters of cells in the gastrocnemius, soleus, and extensor digitorum longus muscles.

FIG. 4D is a series of bar graphs comparing densities of cells in the gastrocnemius, soleus, and extensor digitorum longus muscles.

### DETAILED DESCRIPTION

The disclosed subject matter is a composition and method of treatment to improve exercise tolerance in human patients and animal subjects.

The terms “a” and “an” as used herein mean “one or more” and include the plural unless the context is inappropriate.

Reference to “about” a value or parameter herein includes (and describes) variations that are directed to that value or parameter per se. In embodiments, the term “about” refers to +/−10%, +/−5%, or +/−1%, of the designated value.

The “comprise” or variations such as “comprises” or “comprising,” will be understood to imply the inclusion of a stated element, integer or step, or group of elements, integers or steps, but not the exclusion of any other element, integer or step, or group of elements, integers or steps. Embodiments described herein also include “consisting” and/or “consisting essentially of” aspects.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route.

The phrase “parenteral administration” and “administered parenterally” as used herein means modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

As used herein, the term “composition” or “pharmaceutical composition” refers to the combination of an active agent with an excipient or a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, PA [1975]).

As used herein, the term “pharmaceutically acceptable salt” refers to any circular salt (e.g., acid or base) of a compound of the present invention suitable for pharmaceutical administration which, upon administration to a subject, is capable of providing a compound of this invention or an active metabolite or residue thereof. As is known to those of skill in the art, “salts” of the compounds of the present invention may be derived from inorganic or organic acids and bases.

Examples of acids include, but are not limited to, hydrochloric, hydrobromic, sulfuric, nitric, perchloric, fumaric, maleic, phosphoric, glycolic, lactic, salicylic, succinic, toluene psulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW4+, wherein W is C1-4 alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH⁴⁺, and NW⁴⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The method described herein to improve exercise tolerance comprises administration of RRx-001, which is also called ABDNAZ, with the chemical name 2-bromo-1-(3,3-dinitroazetidin-1-yl)ethan-1-one. RRx-001 is a small cyclic nitro compound that is currently in a Phase 3 clinical trial for the treatment of cancer. It has the following structure:

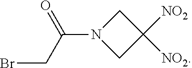 [SMILES: O=C(CBr)N1CC([N+](=O)[O-])([N+](=O)[O-])C1]

A study was conducted to examine whether treatment with the RRx-001 improved exercise and skeletal muscle oxidative capacity in mice. The results, which are summarized below, indicate that the RRx-001 increases various observable criteria to quantify exercise capacity in mice. Accordingly, it is believed that administration of the RRx-001 similarly increases exercise performance and reduces physical fatigue in human patients and other mammal subjects.

### Endurance Study

An endurance exercise capacity (EEC) test was performed on male and female adult mice (n=6 per group). The mice were administered a dose of either 5 mg/kg or 2 mg/kg RRx-001 3 times per week. Subsequent to the administration, the mice were tested until exhaustion on a motorized treadmill.

Fatigue, for the purpose of the study, was defined as the inability of the mice to maintain the appropriate pace despite continuous hand stimulation for 1 minute. The treadmill had a velocity of 30 meters per minute with a 10% incline. Blood samples were collected immediately after the exercise. Malondialdehyde (MDA) and creatine kinase (CK) were determined with a commercial ELISA assay kit. MDA is an indicator of lipid peroxidation and CK is an indicator of muscle damage.

By weeks 2 and 3 of the study, the exercise times at which mice reached fatigue were significantly longer for groups that were administered RRx-001 when compared to a control group. MDA levels, which were taken from gastrocnemius, soleus, and extensor digitorum muscles, were lower in the groups that were administered RRx-001 than control. Similarly, CK levels from blood collected from the gastrocnemius, soleus, and extensor digitorum muscles were lower than control. The study found that mice that were administered RRx-001 were protected against strenuous exercise-induced muscle damage when compared to control.

### Methods for Administering RRx-001

RRx-001, or a pharmaceutically acceptable salt thereof, may be administered to a patient or animal subject through a variety of methods. In various embodiments, the RRx-001, or a pharmaceutically acceptable salt thereof, may be administered through a vehicle, such as DMSO. In various embodiments, the RRx-001, or a pharmaceutically acceptable salt thereof, is administered as an oral rinse. In various embodiments, the RRx-001, or a pharmaceutically acceptable salt thereof, is administered as a drink to be consumed by the patient or animal subject. In various embodiments, the RRx-001, or a pharmaceutically acceptable salt thereof, is administered as an oral pill or tablet. In various embodiments, the RRx-001, or a pharmaceutically acceptable salt thereof, is administered as an intravenous infusion. In various embodiments, the RRx-001, or a pharmaceutically acceptable salt thereof, is administered as a subcutaneous injection. In various embodiments, the RRx-001, or a pharmaceutically acceptable salt thereof, is administered through inhalation. In various embodiments, the RRx-001, or a pharmaceutically acceptable salt thereof, is administered sublingually. In various embodiments, the RRx-001, or a pharmaceutically acceptable salt thereof, is administered rectally.

The term “subject” or “patient”, when used herein, refers to any mammalian subject to be treated by the methods of the present invention and that is being administered the RRx-001, or a pharmaceutically acceptable salt thereof. Such organisms are preferably mammals (e.g., murines, simians, equines, bovines, porcines, canines, felines, and the like), and more preferably humans. The RRx-001, or a pharmaceutically acceptable salt thereof, may be administered to human patients, as well as other mammals such as dogs and horses. In some embodiments, the subject or patient is the non-human mammal. In some embodiments, the non-human subject or patient is the livestock animal. In some embodiments, the non-human subject or patient is the domesticated animal. In some embodiments, the non-human subject or patient is the non-domesticated animal.

### Uses for RRx-001

Fatigue, lack of energy, lack of strength, and lack of physical endurance are common symptoms to a variety of medical conditions. The RRx-001, or a pharmaceutically acceptable salt thereof, may be administered to improve exercise tolerance for many such conditions that result in reduced exercise capacity. Examples of the various medical conditions to which the RRx-001, or a pharmaceutically acceptable salt thereof, may decrease severity of symptoms related to exercise capacity include, but are not limited to, those listed below. In various embodiments, the RRx-001, or a pharmaceutically acceptable salt thereof, may be administered to human patients or other mammal subjects that are suspected of having hypercholesterolemia, hyperlipidemia, cancer, muscular dystrophies, peripheral vascular disease, patent foramen ovale, obesity, type 2 diabetes, angina pectoris; heart failure, mitochondrial disorders or diseases, chronic obstructive pulmonary disease, hyperCKemia, neurodegenerative disease, motor neuron disease, neuromuscular disease, multiple sclerosis, Charcot-Marie-Tooth disease, multiple sclerosis, myositis including polymyositis and dermatomyositis, cardiovascular disease, pulmonary artery hypertension, insulin resistance, hypertension, myoedema, rhabdomyolysis, idiopathic chronic muscle fatigue, reduced skeletal muscle function, disrupted skeletal muscle function or metabolism, cardiac abnormalities, or dysfunctional muscle, dysfunctional heart, or dysfunctional skeletal metabolism.

In certain embodiments, the cancer is a solid tumor. In certain embodiments, the cancer is brain cancer, bladder cancer, breast cancer, cervical cancer, cholangiocarcinoma, colon cancer, colorectal cancer, endometrial cancer, esophageal cancer, lung cancer, liver cancer, melanoma, ovarian cancer, pancreatic cancer, prostate cancer, rectal cancer, renal cancer, stomach cancer, testicular cancer, or uterine cancer. In certain embodiments, the cancer is brain cancer. In certain embodiments, the cancer is colorectal cancer. In certain embodiments, the cancer is cholangiocarcinoma or lung cancer. In certain embodiments, the cancer is lung cancer. In certain embodiments, the lung cancer is small cell lung cancer. In certain other embodiments, the cancer is non-small cell lung cancer. In certain embodiments, the cancer is a leukemia or lymphoma. In certain embodiments, the cancer is a B-cell lymphoma or non-Hodgkin lymphoma.

Additional exemplary cancers include, for example, bladder cancer, breast cancer, cervical cancer, colon cancer colorectal cancer, endometrial cancer, esophageal cancer, leukemia, Jung cancer, liver cancer, melanoma, ovarian cancer, pancreatic cancer, prostate cancer, rectal cancer, renal cancer, stomach cancer, testicular cancer, and uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, bilary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid. cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma. T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In various embodiments, administration of a therapeutically effective amount of the RRx-001, or a pharmaceutically acceptable salt thereof, may result in increased VO₂ max in the patient. VO₂ max, when used herein, refers to a volume of oxygen that is inhaled during exercise. In various embodiments, administration of the effective amount (e.g., the therapeutically effective amount) of the RRx-001, or a pharmaceutically acceptable salt thereof, may result in improved left ventricular (LV) function. Improvement of the LV function may be measured by a variety of criteria including measuring left ventricular ejection fraction, which is a volume of blood pumped out of the heart during systole relative to the volume in the left ventricle at the end of diastole. Left ventricular ejection fraction may be determined by dividing a volume of blood ejected in a single ventricular contraction by end-systolic volume.

In various embodiments, administration of the effective amount (e.g., the therapeutically effective amount) of the RRx-001, or a pharmaceutically acceptable salt thereof, may result in reduced low-density lipoprotein (LDL) levels and increased high-density lipoprotein (HDL) levels. In various embodiments, administration of the therapeutically effective amount of the RRx-001, or the pharmaceutically acceptable salt thereof, may result in decreased serum triglyceride (TAG) levels. In various embodiments, administration of the effective amount (e.g., the therapeutically effective amount) of the RRx-001, or the pharmaceutically acceptable salt thereof, may result in improved fasting glycemia. In various embodiments, administration of the effective amount (e.g., the therapeutically effective amount) of the RRx-001, or a pharmaceutically acceptable salt thereof, may result in improved insulin resistance.

In various embodiments, administration of the effective amount (e.g., the therapeutically effective amount) of the RRx-001, or a pharmaceutically acceptable salt thercof, may result in decreased blood pressure. In various embodiments, administration of the effective amount (e.g., the therapeutically effective amount) of the RRx-001, or a pharmaceutically acceptable salt thereof, may result in delayed onset muscle soreness and pain threshold. In various embodiments, administration of the effective amount (e.g., the therapeutically effective amount) of the RRx-001, or a pharmaceutically acceptable salt thereof, may result in decreased slow skeletal troponin I concentration (sTnI). In various embodiments, administration of the effective amount (e.g., the therapeutically effective amount) of the RRx-001, or a pharmaceutically acceptable salt thereof, may result in decreased serum myoglobin concentration.

In various embodiments, administration of the effective amount (e.g., the therapeutically effective amount) of the RRx-001, or a pharmaceutically acceptable salt thereof. may result in decreased serum IL-1β, IL-6, decreased 8-hydroxy-2′-deoxyguanosine (8-OHdG) concentration. In various embodiments, administration of the effective amount (e.g., the therapeutically effective amount) of the RRx-001, or a pharmaceutically acceptable salt thercof, may result in decreased TNF-α concentration. In various embodiments, administration of the effective amount (e.g., the therapeutically effective amount) of the RRx-001, or a pharmaceutically acceptable salt thereof, may result in reduced sedimentation rate and C-reactive protein. In various embodiments, administration of the effective amount (e.g., the therapeutically effective amount) of the RRx-001, or a pharmaceutically acceptable salt thereof, may result in enhanced fatty acid metabolism, oxidation, and utilization. In various embodiments, administration of the effective amount (e.g., the therapeutically effective amount) of the RRx-001may result in increased long-chain fatty acid (LCFA) muscle uptake.

In various embodiments, peripheral vasodilation occurs in the subject subsequent to administration of the RRx-001, or a pharmaceutically acceptable salt thereof. Peripheral vasodilation, as used herein, may refer to widening of one or more blood vessels. When blood vessels dilate, the flow of blood is increased due to a decrease in vascular resistance and increase in cardiac output. Dilation of arterial blood vessels decreases blood pressure. In some embodiments, the response may be localized to a specific organ (depending on the metabolic needs of a particular tissue, as during strenuous exercise). Vasodilation enables the delivery of extra oxygen and nutrients to the muscles during exercise.

In various embodiments, the human patients who are administered the RRx-001, or a pharmaceutically acceptable salt thereof, are physically active individuals, including, but not limited to, athletes, body builders, and manual workers. In various embodiments, the effective amount (e.g., the therapeutically effective amount) of the RRx-001, or a pharmaceutically acceptable salt thereof, is administered to the human patient prior to said physical activity. In another embodiment, the human patients who are administered the RRx-001, or a pharmaceutically acceptable salt thereof, are individuals who wish to lose weight or enhance their physique. In yet another embodiment, the human patients who are administered the RRx-001, or a pharmaceutically acceptable salt thereof, are healthy human patients.

In various embodiments, the mammalian subjects who are administered the RRx-001, or a pharmaceutically acceptable salt thereof, are dogs or horses. In various embodiments, the mammalian subjects who are administered the RRx-001, or a pharmaceutically acceptable salt thereof, are livestock animals. In certain embodiments, the livestock animals are selected from the group consisting of cows, sheep, goats, and pigs. In other various embodiments, the mammalian subjects who are administered the RRx-001, or a pharmaceutically acceptable salt thereof, are healthy mammals. In certain embodiments, the livestock animals are healthy livestock animals.

### Pharmaceutical Composition

The present disclosure provides compositions or pharmaceutical compositions for enhancing physical performance in a subject. As a general matter, the pharmaceutical composition contains at least one active agent and a pharmaceutically acceptable carrier. In some embodiments, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption). boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders a compound of the present invention orally bioavailable.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and clixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by cither providing a rate controlling membrane or dispersing the compound in a polymer matrix or gel.

Ophthalmic formulations, eye ointments, powders, solutions and the like, are also contemplated as being within the scope of this invention.

In certain embodiments, it may be desirable to introduce the compositions disclosed herein into the central nervous system by any suitable route, including intraventricular, intrathecal and epidural injection. Intraventricular injection may be facilitated by an intraventricular catheter, for example, attached to a reservoir, such as an Ommaya reservoir.

In some embodiments, the pharmaceutical composition is configured as an inhalable formulation. In some embodiments, the inhalable formulation is configured as a dosage form adapted for pulmonary or nasal administration to the subject. In some embodiments, for example, dosage forms may include those adapted for inhalation such as aerosols and dry powders. In some embodiments, the formulation described herein is suitable for topical delivery to the lung via nose inhalation and/or mouth inhalation. In other embodiments, the compositions disclosed herein may also be administered directly to the lung by inhalation by a number of different devices.

In some embodiments, the inhalable formulation is configured as an aerosol formulation that comprises a propellant. In some embodiments, the propellant can provide energy to deliver molecules of any of the compounds described herein to the lung. Representative propellants are disclosed in U.S. Pat. Nos. 6,932,962 B1 and 8,367,734 B1. In some embodiments, the propellant is presented in the aerosol formulation in an amount ranging from 98% to 99% (w/w) relative to the total weight of the aerosol formulation.

In some embodiments, the aerosol formulation further comprises a surfactant, a co-solvent, and/or a pH buffer. The surfactant can give fine dispersions of the compounds described herein in the propellant and can stabilize the mixture of the compounds described herein in the propellant. In some embodiments, the surfactant comprises a fatty acid or a pharmaceutically acceptable salt thereof, a bile salt, a phospholipid, or an alkyl saccharide. In some embodiments, the surfactant is presented in the formulations described herein in an amount of less than 5% (w/w) (e.g., less than 4%, less than 3%, less than 2%, less than 1% by weight) relative to the total weight of the aerosol formulation.

In some embodiments, the co-solvent can help to stabilize the surfactant and improve the dispersion characteristics. In some embodiments, exemplary co-solvents include ethyl alcohol, isopropyl alcohol, propylene glycol, ethylene glycol, propane, butane, isobutane, pentane, dimethyl ether, diethyl ether and the like. In some embodiments, the co-solvent is present in the formulation in an amount ranging from 0.5% to 20% w/w of the total weight of the formulation. In some embodiments, the co-solvent is present in the formulation in an amount ranging from 0.5% to 5% w/w of the total weight of the formulation. In some embodiments, the co-solvent is present in the formulation in an amount ranging from 0.5% to 1.5% (w/w) of the total weight of the formulation. Representative surfactants, co-solvents, and pH buffers are disclosed in U.S. Pat. Nos. 6,932,962 B1 and 8,367,734 B1.

In some embodiments, provided herein are combinations containing the aerosol formulation with the propellant and a pressurized bottle or a nebulizer. In some embodiments, the aerosol formulation with the propellant may be packed in pressurized bottles, where a dosage controller may be used with the pressurized bottle to control the amount of drug being administrated in each spray. In some embodiments, the aerosol formulation with the propellant may be packed in pressurized bottles with a dosage controller, where the dosage controller comprises a valve that controls the delivery of a metered amount of the drug.

In some embodiments, the aerosol formulation is propellant-free and comprises the effective amount of the RRx-001 or the pharmaceutical composition and a solvent. In some embodiments, exemplary solvents include water and alcohols, such as ethanol, isopropanol, and glycols, such as propylene glycol, polyethylene glycol, polypropylene glycol, glycol ether, glycerol and polyoxyethylene alcohols. In some embodiments, the solvent is present in the propellant-free aerosol formulation in an amount ranging from about 0.01% to about 90% (w/w), or about 0.01% to about 50% (w/w), or about 0.01% to about 25% (w/w), or about 0.01% to about 10% (w/w), or about 0.01% to about 5% (w/w) relative to the total weight of the aerosol formulation.

In some embodiments, the propellant-free aerosol formulation may further comprise an emulsifying agent. In some embodiments, exemplary emulsifying agents are disclosed in U.S. Pat. No. 9,498,437 B2. In some embodiments, the emulsifying agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a complexing agent. In some embodiments, exemplary complexing agents include, but are not limited to, ethylenediaminetetraacetic acid (EDTA) or a pharmaceutically acceptable salt thereof. such as the disodium salt, citric acid, nitrilotriacetic acid and the salts thereof, and sodium edetate. Representative complexing agents are disclosed in U.S. Pat. No. 9,498,437 B2. In some embodiments, the complexing agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a tonicity agent that can adjust the isotonicity of the present formulations. In some embodiments, exemplary tonicity agents include, but are not limited to, sodium chloride, potassium chloride, zinc chloride, calcium chloride or mixtures thereof. Other osmotic adjusting agents may also include, but are not limited to, mannitol, glycerol, and dextrose or mixtures thereof.

Representative tonicity agents are disclosed in U.S. Pat. No. 9,498,437 B2. In some embodiments, the tonicity agent is present in the propellant-free aerosol formulations in an amount ranging from about 0.01% to about 10% (w/w), or about 1% to about 10% (w/w), or about 1% to about 6% (w/w) relative to the total weight of the aerosol formulation. In some embodiments, the aerosol formulation may further comprise the pH buffer.

In some embodiments, provided herein are combinations containing the propellent-free aerosol formulation provided herein and a nebulizer. In some embodiments, the nebulizer can nebulize liquid formulations, including the propellant-free aerosol formulations detailed herein, and produce a nebulized aerosol mist. In some embodiments, the nebulizer may further have an internal baffle, which can selectively remove large droplets from the mist by impaction and allow the droplets to return to the reservoir, so that only fine aerosol droplets are entrained into the lung of the subject by the inhaling air/oxygen. Examples of nebulizers include devices supplied by Sheffield Pharmaceuticals, St. Louis, MO. (Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus, OH).

In some embodiments, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver the RRx-001 and/or pharmaceutical compositions thereof directly to the lung. Specifically, the MDI comprises an aerosol container suitable for containing a propellant-based aerosol formulation and/or a metering valve, for example a side valve, which controls the release of the aerosol formulation to the subject. Representative methods and devices to administer the aerosol formulation with the propellant are disclosed in U.S. Pat. No. 9,498,437 B2.

In another embodiment, a Dry Powder Inhaler (“DPI”') device may be used to administer the compositions disclosed herein to the lung. DPI devices typically use a mechanism such as a burst of gas to create a cloud of dry powder inside a container, which may then be inhaled by the patient and are well known in the art. In a particular embodiment, a popular variation is the multiple dose DPI (“MDDPI”) system, which allows for the delivery of more than one therapeutic dose. MDDPI devices are commercially available from a number of pharmaceutical companies e.g., Schering Plough, Madison, NJ). For example, capsules and cartridges of gelatin for use in an inhaler or insufflator may be formulated containing a powder mix of the compositions disclosed herein and a suitable powder base such as lactose or starch for these systems.

In some embodiments, another type of device that may be used to deliver the compositions disclosed herein to the lung is a liquid spray device supplied, for example, by Aradigm Corporation, Hayward, CA. Liquid spray systems use extremely small nozzle holes to aerosolize liquid drug formulations that may then be directly inhaled into the lung.

In some embodiments, a nebulizer is used to deliver the compositions disclosed herein to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96). Examples of nebulizers include devices supplied by Sheffield Pharmaceuticals, St. Louis, MO. (Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus, OH).

In other embodiments, an electrohydrodynamic (“EHD”) aerosol device is used to deliver the compositions disclosed herein to the lung of a patient. EHD aerosol devices use electrical energy to aerosolize liquid drug solutions or suspensions (see e.g., Noakes et al., U.S. Pat. No. 4,765,539). The electrochemical properties of the formulation may be important parameters to optimize when delivering the RRx-001 and/or pharmaceutical composition thereof to the lung with an EHD aerosol device. EHD aerosol devices may more efficiently deliver drugs to the lung than existing pulmonary delivery technologies.

Suitable pharmaceutically acceptable excipients will vary depending upon the particular dosage form chosen. In some embodiments, for example, certain pharmaceutically acceptable excipients may be chosen for their ability to: facilitate the production of aerosol for inhalation. facilitate the production of solution or mist for inhalation, facilitate the production of dry powder for inhalation, or facilitate the production of stable dosage forms.

In some embodiments, the compositions disclosed herein can be delivered via sustained release systems, e.g., oral sustained release systems. In other embodiments, a pump may be used (e.g., Langer, supra, Sefton, 1987, CRC Crit. Ref Biomed. Eng. 14:201; Saudek et al., 1989, N. Engl. J Med. 321:574).

In some embodiments, polymeric materials can be used (e.g., “Medical Applications of Controlled Release,” Langer and Wise (eds.), CRC Press, Boca Raton. Florida (1974); “Controlled Drug Bioavailability,” Drug Product Design and Performance, Smolen and Ball (eds.), Wiley, New York (1984); Ranger et al., 1983. J Macromol. Sci. Rev. Macromol Chem. 23:61; Levy et al., 1985, Science 228:190; During et al., 1989, Ann. Neurol. 25:351; Howard et al., 1989, J. Neurosurg. 71:105).

In other embodiments, polymeric materials are used for oral sustained release delivery. Polymers include, but are not limited to, sodium carboxymethylcellulose, hydroxypropylcellulose, hydroxypropylmethylcellulose and hydroxyethylcellulose (most preferred, hydroxypropyl methylcellulose). Other cellulose ethers have been described (Alderman, Int. J. Pharm. Tech. & Prod. Mfr. 1984, 5(3) 1-9). Factors affecting drug release are well known to the skilled artisan and have been described in the art (Bamba et al., Int. J. Pharm. 1979, 2, 307).

In other embodiments, enteric-coated preparations can be used for oral sustained release administration. Coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In other embodiments, osmotic delivery systems are used for oral sustained release administration (Verma et al., Drug Dev. Ind. Pharm., 2000, 26:695-708). In some embodiments, OROS™ osmotic devices are used for oral sustained release delivery devices (Theeuwes et al., U.S. Pat. No. 3,845,770; Theeuwes et al., U.S. Pat. No. 3,916,899).

In yet other embodiments, a controlled-release system can be placed in proximity of the target of RRx-001 described herein and/or pharmaceutical composition, thus requiring only a fraction of the systemic dose (e.g., Goodson, in “Medical Applications of Controlled Release,” supra, vol. 2, pp. 115-138 (1984)). Other controlled-release systems previously may also be used (Langer, 1990, Science 249:1527-1533).

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In some cases, in order to prolong the effect of a drug, it is desirable to slow the absorption of the drug administered by subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

When the compounds of the present invention are administered as pharmaceuticals to subjects, they can be given per se or as a pharmaceutical composition containing, for example, 0.1 to 99% (more preferably, 10 to 30%) of active ingredient in combination with a pharmaceutically acceptable carrier.

### RRx-001 Administration with One or More Agents and/or a Blood Product

The RRx-001, or a pharmaceutically acceptable salt thereof, may be administered concurrently with one or more agents. In various embodiments, the one or more other agents include antioxidants, vitamins, minerals, hormones, drugs, compositions, biologicals, or the like. In various embodiments, the RRx-001, or a pharmaceutically acceptable salt thereof, may be administered with one or more antioxidants including, but not limited to lycopene, coenzyme Q10, melatonin, selenium, alpha-lipoic acid, ellagic acid, lutein, resveratrol, anthocyanins, ellagitannins, polyphenols, quercetin, and curcumin.

In various embodiments, the RRx-001, or a pharmaceutically acceptable salt thereof, may be administered with one or more vitamins. The vitamins may include but are not limited to Vitamin A, Vitamin C, Vitamin E, folic acid, and biopterin. In various embodiments, the RRx-001 may be administered with one or more minerals. The one or more minerals may include but are not limited to calcium, phosphorus, potassium, sodium, chloride, magnesium, iron, zinc, iodine, chromium, copper, fluoride, molybdenum, and manganese.

In various embodiments, the one or more agents may comprise a corticosteroid. In various embodiments, the one or more agents may comprise human growth hormone. In various embodiments, the one or more agents may comprise an anabolic steroid. In various embodiments, the one or more agents may comprise levothyroxine. In various embodiments, the one or more agents may comprise erythropoietin. In various embodiments, the one or more agents may comprise selective androgen receptor modulator (SARM). In various embodiments, the one or more agents may comprise human chorionic gonadotrophin (HCG). In various embodiments, the one or more agents may comprise adrenocorticotrophin (ACTH). In various embodiments, the one or more agents may comprise insulin. In various embodiments, the one or more agents may comprise beta 2 agonist. In various embodiments, the one or more agents may comprise marijuana. In various embodiments, the one or more agents may comprise a diuretic. In various embodiments, the one or more agents may comprise a method or substance that causes blood doping. Blood doping, as used herein, may refer to agents or methods that increase a number of circulating red blood cells.

In various embodiments, the one or more agents may comprise a narcotic. In various embodiments, the one or more agents may comprise a beta blocker. In various embodiments, the one or more agents may comprise a nitrite and/or nitrate.

In various embodiments, the one or more agents may comprise a nitric oxide donor, organic nitrates (e.g., glyceryl trinitrate, isosorbide dinitrate), sodium nitroprusside, sydnonimines (e.g., molsidomine, SIN-1), S-nitrosothiols (e.g., s-nitrosoglutathione, SNAP), and NONOates (e.g., spermine NONOate, DETA-NONOate). In various embodiments, the one or more agents may comprise a PDE5 inhibitor such as sildenafil (Viagra), tadalafil (Cialis), vardenafil (Levitra), or avanafil (Stendra). In various embodiments, the one or more agents may comprise a sympathomimetic drug such as dobutamine, albuterol, phenylpropanolamine, amphetamine, and ephedrine, and combinations thereof.

In some embodiments, the pharmaceutical composition may comprise (1) the effective amount of the RRx-001, or a pharmaceutically acceptable salt thereof and (2) at least one of the blood product and the one or more other agents. In some embodiments, the blood product comprises erythrocyte cells. In some embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In some embodiments, the blood product is a mixture of packed red blood cells. In other embodiments, the blood product is whole blood. In some embodiments, the whole blood is autologous whole blood.

Concurrent administration, as used herein, may refer to administering the RRx-001, or a pharmaceutically acceptable salt thereof, over a period of time that overlaps with administration of the one or more other agents and/or the blood product. For example, the RRx-001, or a pharmaceutically acceptable salt thereof, may be administered a frequency of once per week for a month while another agent and/or the blood product is administered at regular intervals over the same period. In various embodiments, the one or more other agents and/or the blood product are included with the administration of the RRx-001, or a pharmaceutically acceptable salt thereof. For example, the one or more other agents and/or the blood product are combined with RRx-001 prior to administrating the RRx-001, or a pharmaceutically acceptable salt thereof.

In some embodiments, the one or more other agents in the pharmaceutical composition are subject to a reduced incidence of drug-drug interaction as compared to direct administration of the same one or more other agents at the same dose without being mixed with the blood product prior to administration. In certain embodiments, the reduced incidence of drug-drug interaction permits the use of a second agent that would have otherwise been contraindicated.

The present invention can provide methods of attenuating interactions of a first drug (e.g., a first therapeutic agent) and a second drug (e.g., a second therapeutic agent) in a mammal. As described herein, interactions of drugs, or drug-drug interactions, can refer to the changes of the effects of a drug or a pharmaceutical composition on a mammal when the pharmaceutical composition is taken together with a second drug or second pharmaceutical composition. In some embodiments, the interactions can occur when more than two drugs are concurrently in a mammal, regardless of the time between the administrations of the two or more drugs and thereby, and react with each other.

In some embodiments, as described herein, “attenuating interactions” of drugs refers to actions that result in reducing or preventing any types of interactions between two or more drugs or reducing the hypersensitivity, the toxicity, or adverse effects that are caused by the interactions of two or more drugs. In some embodiments, the interactions can include, but are not limited to, synergistic or antagonistic interactions. By way of examples, attenuating interactions of the drugs can be at least any one of the following scenarios: reducing and/or preventing drug-drug physical interactions, reducing and/or preventing drug-drug pharmacokinetic interactions, reducing and/or preventing the hypersensitivity caused by co-existence of the drugs, reducing and/or preventing the toxicity caused by co-existence of drugs, or reducing and/or preventing the antagonistic interactions of drugs.

In some embodiments, the effects of the attenuated interactions can be delayed, decreased, or enhanced absorption of either pharmaceutical composition, and thereby decreases or increases the action of the one or more other agents or the pharmaceutical composition. In some embodiments, the attenuated interactions can impact the transport or the distribution of the one or more other agents or the pharmaceutical compositions.

Accordingly, in certain embodiments, the subject has reduced incidence and/or severity of side effects compared to subjects receiving a direct administration of the same one or more other agents at the same dose without being mixed with the blood product prior to administration. In certain embodiments, the subject has reduced side effects compared to subjects receiving a direct administration of the same one or more other agents at the same dose without being mixed with the blood product prior to administration. In certain embodiments, the dose of the one or more other agents in the pharmaceutical composition is at least about 10% to about 300% more than the dose recommended for a direct administration of the same one or more other agents without being mixed with the blood product prior to administration. In certain embodiments, the dose of the one or more other agents in the pharmaceutical composition is at least 1%, at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, or higher, inclusive of all ranges and subranges therebetween, more than the dose recommended for a direct administration of the same one or more other agents without being mixed with the blood product prior to administration.

In certain embodiments, the one or more other agents have a longer circulating half-life in the subject compared to direct administration of the same one or more other agents at the same dose without being mixed with the blood product prior to administration. In certain embodiments, the circulating half-life of the one or more other agents is at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 110%, 110%, 120%, 130%, 140%, 150%, 160%, 170%, 180%, 190%, 200%, 250%, 300%, 350%, 400%, 450%, 500%, 600%, 700%, 800%, 900%, 1000%, or more, longer than the circulating half-life of the same one or more other agents at the same dose without being mixed with the blood product before administration.

### Amount of RRx-001

The RRx-001, or a pharmaceutically acceptable salt thereof, may be administered to a patient in various dosages at various frequencies +over various periods of time. The dosages provided herein refer to the amount of the RRx-001, excluding the weight of any counterion that may be present. Each of the dosage, frequency of dosage, and period of time to which the dosage is administered may be adjusted based on various factors including but not limited to the medical condition of the patient, the desired outcome, and the one or more (if any) agents that are administered concurrently with the RRx-001, or a pharmaceutically acceptable salt thereof.

In various embodiments, the dosage may be in units of a mass of the RRx-001, volume of the RRx-001, mass of the RRx-001 to mass of the patient, mass of the RRx-001 to volume of the patient, volume of the RRx-001 to volume of the patient, mass of the RRx-001 to surface area of the patient, volume of the RRx-001 to surface area of the patient, or the like. In various embodiments, the RRx-001 may be administered as the RRx-001, or a pharmaceutically acceptable salt thereof. In various embodiments, the RRx-001, or a pharmaceutically acceptable salt thereof, may be administered in one or more doses of between about 0.5 mg and 200 mg. In various embodiments, the RRx-001, or a pharmaceutically acceptable salt thereof, may be administered in one or more doses of between about 0.1 mg and 0.5 mg. In various embodiments, the RRx-001, or a pharmaceutically acceptable salt thereof, may be administered in one or more doses of between about 0.5 mg and 1.0 mg. In various embodiments, the RRx-001, or a pharmaceutically acceptable salt thereof, may be administered in one or more doses of between about 1.0 mg and 1.5 mg. In various embodiments, the RRx-001, or a pharmaceutically acceptable salt thereof, may be administered in one or more doses of between about 1.5 mg and 2.0 mg. In various embodiments, the RRx-001, or a pharmaceutically acceptable salt thereof, may be administered in one or more doses of between about 2.0 mg and 2.5 mg. In various embodiments, the RRx-001, or a pharmaceutically acceptable salt thereof, may be administered in one or more doses of between about 2.5 mg and 3.0 mg. In various embodiments, the RRx-001, or a pharmaceutically acceptable salt thereof, may be administered in one or more doses of between about 3.0 mg and 3.5 mg. In various embodiments, the RRx-001, or a pharmaceutically acceptable salt thereof, may be administered in one or more doses of between about 3.5 mg and 4.0 mg. In various embodiments, the RRx-001, or a pharmaceutically acceptable salt thereof, may be administered in one or more doses of between about 4.0 mg and 4.5 mg. In various embodiments, the RRx-001, or a pharmaceutically acceptable salt thereof, may be administered in one or more doses of between about 4.5 mg and 5.0 mg. In various embodiments, the RRx-001, or a pharmaceutically acceptable salt thereof, may be administered in one or more doses of between about 5 mg and 6 mg. In various embodiments, the RRx-001, or a pharmaceutically acceptable salt thereof, may be administered in one or more doses of between about 6 mg and 7 mg. In various embodiments, the RRx-001, or a pharmaceutically acceptable salt thereof, may be administered in one or more doses of between about 7 mg and 8 mg. In various embodiments, the RRx-001, or a pharmaceutically acceptable salt thereof, may be administered in one or more doses of between about 8 mg and 9 mg. In various embodiments, the RRx-001, or a pharmaceutically acceptable salt thereof, may be administered in one or more doses of between about 9 mg and 10 mg.

In various embodiments, the RRx-001, or a pharmaceutically acceptable salt thereof, may be administered in one or more doses of between about 10 mg and 15 mg. In various embodiments, the RRx-001, or a pharmaceutically acceptable salt thereof, may be administered in one or more doses of between about 15 mg and 20 mg. In various embodiments, the RRx-001, or a pharmaceutically acceptable salt thereof, may be administered in one or more doses of between about 20 mg and 25 mg. In various embodiments, the RRx-001, or a pharmaceutically acceptable salt thereof, may be administered in one or more doses of between about 25 mg and 30 mg. In various embodiments, the RRx-001, or a pharmaceutically acceptable salt thereof, may be administered in one or more doses of between about 30 mg and 35 mg. In various embodiments, the RRx-001, or a pharmaceutically acceptable salt thereof, may be administered in one or more doses of between about 35 mg and 40 mg. In various embodiments, the RRx-001, or a pharmaceutically acceptable salt thereof, may be administered in one or more doses of between about 40 mg and 45 mg. In various embodiments, the RRx-001, or a pharmaceutically acceptable salt thereof, may be administered in one or more doses of between about 45 mg and 50 mg. In various embodiments, the RRx-001, or a pharmaceutically acceptable salt thereof, may be administered in one or more doses of between about 50 mg and 55 mg. In various embodiments, the RRx-001, or a pharmaceutically acceptable salt thereof, may be administered in one or more doses of between about 55 mg and 60 mg. In various embodiments, the RRx-001, or a pharmaceutically acceptable salt thereof, may be administered in one or more doses of between about 60 mg and 65 mg. In various embodiments, the RRx-001, or a pharmaceutically acceptable salt thereof, may be administered in one or more doses of between about 65 mg and 70 mg. In various embodiments, the RRx-001, or a pharmaceutically acceptable salt thereof, may be administered in one or more doses of between about 70 mg and 75 mg. In various embodiments, the RRx-001, or a pharmaceutically acceptable salt thereof, may be administered in one or more doses of between about 75 mg and 80 mg. In various embodiments, the RRx-001, or a pharmaceutically acceptable salt thereof, may be administered in one or more doses of between about 80 mg and 85 mg. In various embodiments, the RRx-001, or a pharmaceutically acceptable salt thereof, may be administered in one or more doses of between about 85 mg and 90 mg. In various embodiments, the RRx-001, or a pharmaceutically acceptable salt thereof, may be administered in one or more doses of between about 90 mg and 95 mg. In various embodiments, the RRx-001, or a pharmaceutically acceptable salt thereof, may be administered in one or more doses of between about 95 mg and 100 mg.

In various embodiments, the RRx-001, or a pharmaceutically acceptable salt thereof. may be administered in one or more doses of between about 100 mg and 110 mg. In various embodiments, the RRx-001, or a pharmaceutically acceptable salt thereof, may be administered in one or more doses of between about 110 mg and 120 mg. In various embodiments, the RRx-001, or a pharmaceutically acceptable salt thereof, may be administered in one or more doses of between about 120 mg and 130 mg. In various embodiments, the RRx-001, or a pharmaceutically acceptable salt thereof, may be administered in one or more doses of between about 130 mg and 140 mg. In various embodiments, the RRx-001, or a pharmaceutically acceptable salt thereof, may be administered in one or more doses of between about 140 mg and 150 mg. In various embodiments, the RRx-001, or a pharmaceutically acceptable salt thereof, may be administered in one or more doses of between about 150 mg and 160 mg. In various embodiments, the RRx-001, or a pharmaceutically acceptable salt thereof, may be administered in one or more doses of between about 160 mg and 170 mg. In various embodiments, the RRx-001, or a pharmaceutically acceptable salt thereof, may be administered in one or more doses of between about 170 mg and 180 mg. In various embodiments, the RRx-001, or a pharmaceutically acceptable salt thereof, may be administered in one or more doses of between about 180 mg and 190 mg. In various embodiments, the RRx-001, or a pharmaceutically acceptable salt thereof, may be administered in one or more doses of between about 190 mg and 200 mg.

In various embodiments, the RRx-001, or a pharmaceutically acceptable salt thereof, may be administered in one or more doses of between about 0.5 mg and 10 mg. In various embodiments, the RRx-001, or a pharmaceutically acceptable salt thereof, may be administered in one or more doses of between about 10 mg and 50 mg. In various embodiments, the RRx-001, or a pharmaceutically acceptable salt thereof, may be administered in one or more doses of between about 50 mg and 100 mg. In various embodiments, the RRx-001, or a pharmaceutically acceptable salt thereof, may be administered in one or more doses of between about 100 mg and 150 mg. In various embodiments, the RRx-001, or a pharmaceutically acceptable salt thereof, may be administered in one or more doses of between about 150 mg and 200 mg.

### Frequency

Each dose of the RRx-001, or a pharmaceutically acceptable salt thereof, may be administered at various intervals over a time period until the end of a dosing period. The various intervals are referred to herein as a frequency. In various embodiments, the RRx-001, or a pharmaceutically acceptable salt thereof, may be administered once per day, once per week, twice per week, once per month, once every 3 months, or combinations thereof. In an exemplary embodiment, each dose of the RRx-001, or a pharmaceutically acceptable salt thereof, is administered at a frequency of about 1 dose per 24 hours. In an exemplary embodiment, each dose of the RRx-001, or a pharmaceutically acceptable salt thereof, is administered at a frequency of about 3 doses per week. In an exemplary embodiment, each dose of the RRx-001, or a pharmaceutically acceptable salt thereof, is administered at a frequency of about 2 doses per week. In an exemplary embodiment, each dose of the RRx-001, or a pharmaceutically acceptable salt thereof, is administered at a frequency of about 1 dose per week. In an exemplary embodiment, each dose of the RRx-001, or a pharmaceutically acceptable salt thereof, is administered at a frequency of about 2 doses per month. In an exemplary embodiment, each dose of the RRx-001, or a pharmaceutically acceptable salt thereof, is administered at a frequency of about 1 dose per month. In an exemplary embodiment, each dose of the RRx-001, or a pharmaceutically acceptable salt thereof, is administered at a frequency of about 1 dose per 2 months. In an exemplary embodiment, each dose of the RRx-001, or a pharmaceutically acceptable salt thereof, is administered at a frequency of about 1 dose per 3 months.

In an exemplary embodiment, each dose of the RRx-001, or a pharmaceutically acceptable salt thereof, is administered at a frequency of between about 1 dose per day and about 1 dose per week until the end of a dosing period. In an exemplary embodiment, each dose of the RRx-001, or a pharmaceutically acceptable salt thereof, is administered at a frequency of between about 1 dose per day and about 2 doses per week until the end of a dosing period. In an exemplary embodiment, each dose of the RRx-001, or a pharmaceutically acceptable salt thereof, is administered at a frequency of between about 1 dose per week and about 1 dose per month until the end of a dosing period. In an exemplary embodiment, each dose of the RRx-001, or a pharmaceutically acceptable salt thereof, is administered at a frequency of between about 1 dose per month and about 1 dose per 3 months until the end of a dosing period.

### Timing of Dose before Physical Performance

The disclosed subject matter increases exercise capacity subsequent to administration of the RRx-001, or a pharmaceutically acceptable salt thereof. In various embodiments, the exercise capacity comprises one or more physical performances. The term “physical performance”, when used herein, refers to any physical activity performed by a patient that gencrates stress for one or more measurable processes in the patient. The RRx-001, or a pharmaceutically acceptable salt thereof, may be administered at various times before the physical performance. For example, the patient may perform the physical performance at any time while the patient is receiving doses of the RRx-001, or a pharmaceutically acceptable salt thereof, at a regular frequency. In various embodiments, a dose of the RRx-001, or a pharmaceutically acceptable salt thereof, is administered approximately 24 hours before the physical performance. In various embodiments, a dose of the RRx-001, or a pharmaceutically acceptable salt thereof, is administered approximately 48 hours before the physical performance. In various embodiments, a dose of the RRx-001, or a pharmaceutically acceptable salt thereof, is administered approximately 72 hours before the physical performance. In various embodiments, a dose of the RRx-001, or a pharmaceutically acceptable salt thereof, is administered approximately 1 week before the physical performance. In various embodiments, a dose of the RRx-001, or a pharmaceutically acceptable salt thereof, is administered approximately 2 weeks before the physical performance. In various embodiments, a dose of the RRx-001, or a pharmaceutically acceptable salt thereof, is administered approximately 1 month before the physical performance. In various embodiments, a dose of the RRx-001, or a pharmaceutically acceptable salt thereof, is administered any time before the physical performance.

### EXAMPLE

In order that this disclosure may be more fully understood, the following Example is set forth. It should be understood that this Example is for illustrative purposes only and are not to be construed as limiting this disclosure in any manner.

An EEC test was performed on male and female adult mice (n=6 per group). The mice were administered a dose of either 5 mg/kg or 2 mg/kg of the RRx-001 3 times per week. Subsequent to the administration of the RRx-001, the mice were tested until exhaustion on the motorized treadmill. The fatigue, for the purpose of the study, was defined as the inability of the mice to maintain an appropriate pace despite continuous hand stimulation for 1 minute. The treadmill had a velocity of 30 meters per minute with a 10% incline. The blood samples were collected immediately after the exercise. Both MDA and CK were determined with a commercial ELISA assay kit. The MDA is an indicator of lipid peroxidation and CK is an indicator of muscle damage. By weeks 2 and 3 of the study, the exercise times at which mice reached fatigue were significantly longer for groups that were administered the RRx-001 when compared to a control group. The MDA levels, which were taken from gastrocnemius, soleus, and extensor digitorum muscles, were lower in the groups that were administered the RRx-001 than control. Similarly, the CK levels from blood collected from the gastrocnemius, soleus, and extensor digitorum muscles were lower than control. The study found that mice that were administered the RRx-001 were protected against strenuous exercise-induced muscle damage when compared to control.

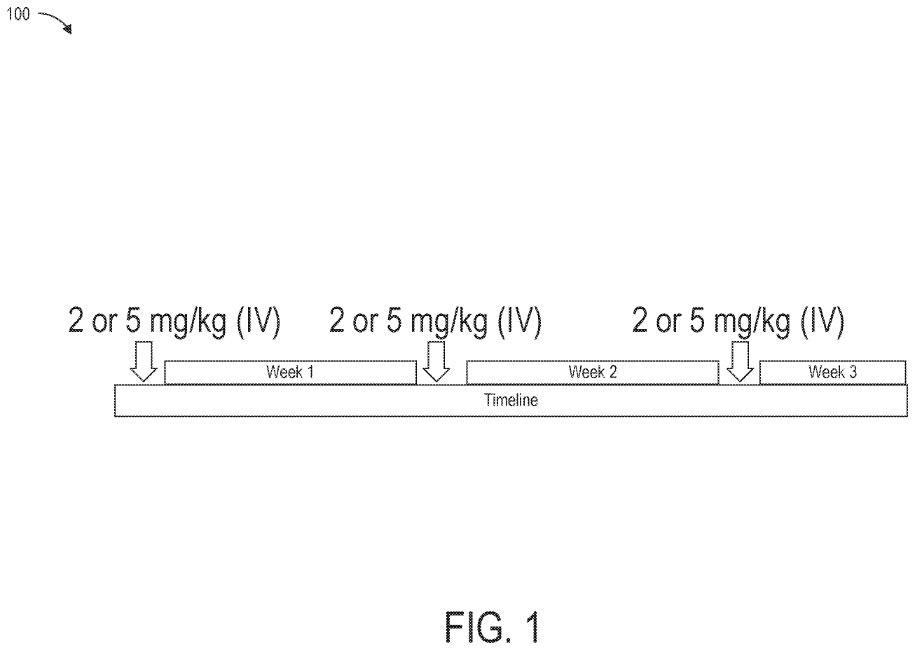

Referring to FIG. 1, FIG. 1 is a schematic 100 of the study design to test changes in exercise capacity in mice subsequent to administration of the RRx-001. For the study, 18 (9 male, and 9 female) adult C57BL/6J mice, 19-23 g, 7-8 wk old (Charles River, King of Prussia, PA) were separated into three groups and treated as follows:
6 mice (3 M, 3 F) received vehicle dimethylsulfoxide (DMSO) intraperitoneally 2 days prior to the start of forced running for 3 weeks.
6 mice (3 M, 3 F) received 2 mg/kg RRx-001 in DMSO intraperitoneally 2 days prior to the start of forced running for 3 weeks.
6 mice (3 M, 3 F) received 2 mg/kg RRx-001 in DMSO intraperitoneally 2 days prior to the start of forced running for 3 weeks

An endurance test was set whereby the mice ran on a motorized treadmill at a speed of 30 meters per minute on a 10% incline until exhaustion. Total running time was recorded to evaluate their performance.

At the conclusion of the experiment, mice were anesthetized with pentobarbital sodium euthanized, and then blood samples were collected via abdominal aorta puncture and the serum was isolated. The serum samples were then stored at −80° C, until required for the creatine kinase (CK) analyses. The gastrocnemius, soleus, and extensor digitorum longus muscles were also removed, washed with physiological saline, and frozen in liquid nitrogen for storage at −80° C. until required for the malondialdehyde (MDA) analysis. All the measurements used the commercial kits that were carried out in accordance with the instructions for each kit from manufacturers.

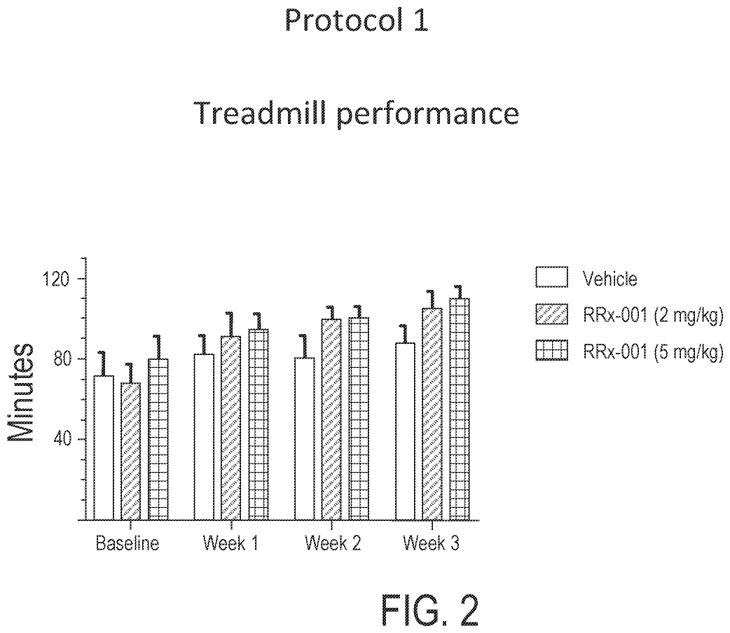

FIG. 2 shows a comparison of exercise mice treated with the RRx-001 and vehicle (control) for three consecutive weeks. The data show that mice in all groups, including the vehicle, increased exercise performance over the course of the study. However, exercise performance for mice in the RRx-001 groups (e.g., 2 mg/kg and 5 mg/kg of the RRx-001) had a greater increase in exercise performance over the 3-week period when compared to the vehicle group. The increased performance for the RRx-001 groups is potentially indicative of an antifatigue effect due to administration of the RRx-001.

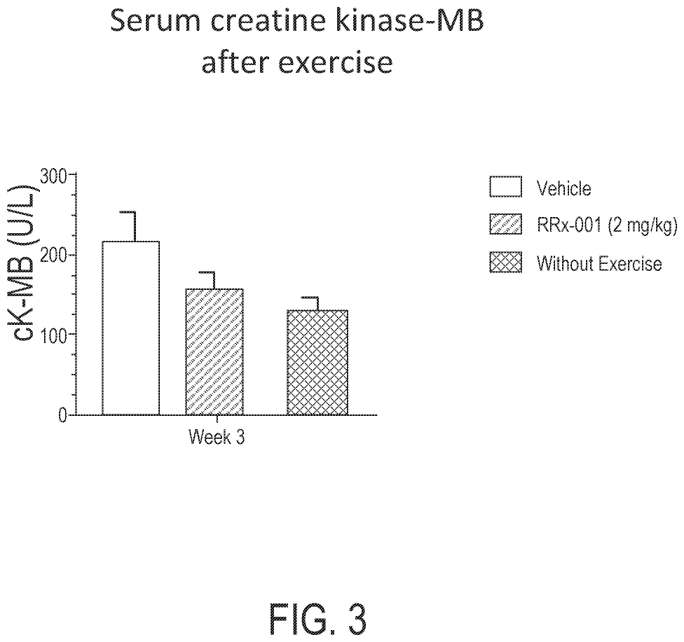

FIG. 3 shows a comparison of levels of creatine kinase-MB (CK-MB) found in mice after exercise. Serum levels of CK were used to estimate exhaustive exercise-induced oxidative muscle damage in the mice. As shown in FIG. 3, the mice that were treated with the RRx-001 had lower levels of the CK-MB than the vehicle. Mice that went without exercise had the lowest levels of the CK-MB.

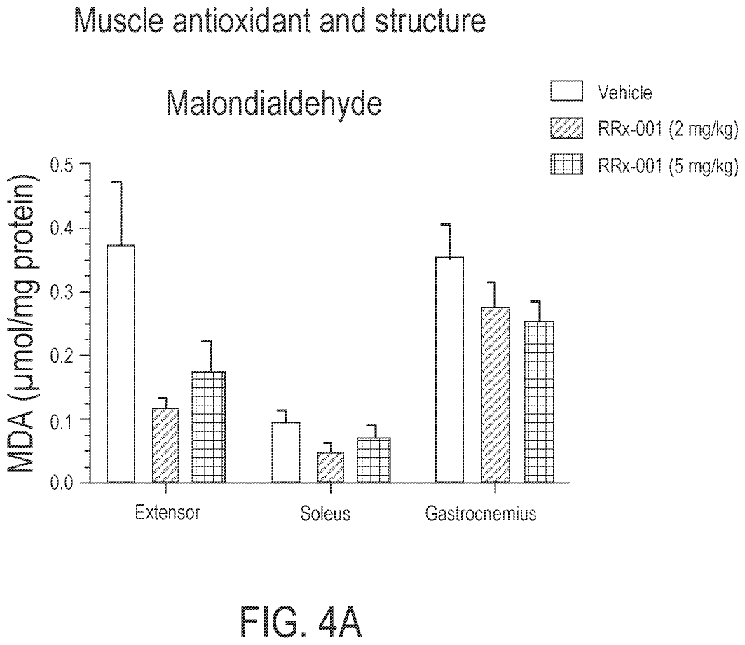

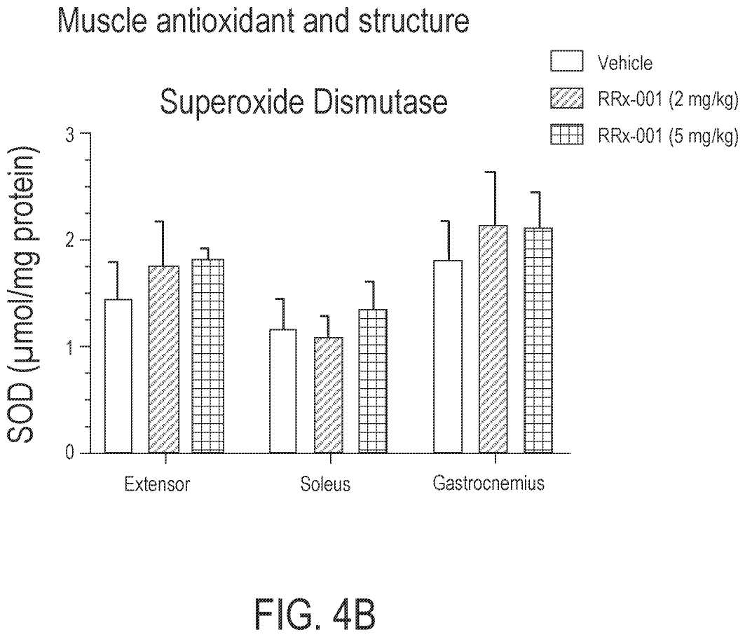

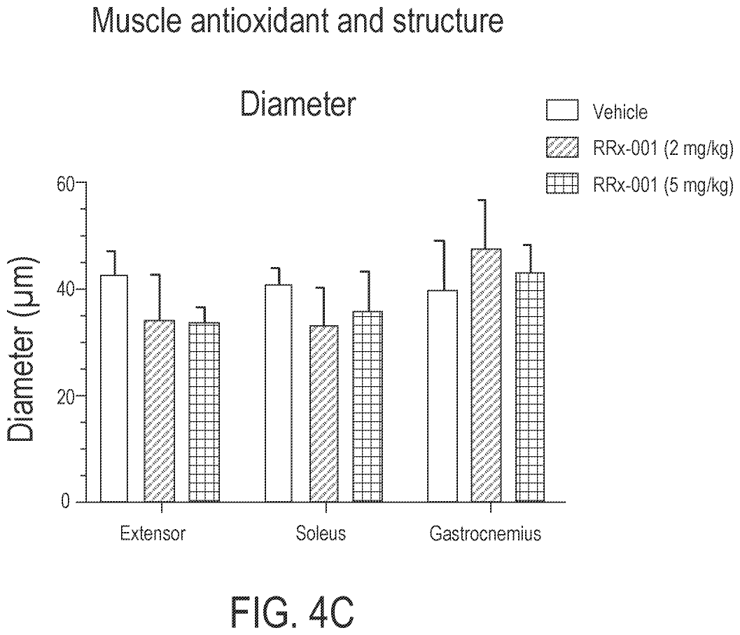

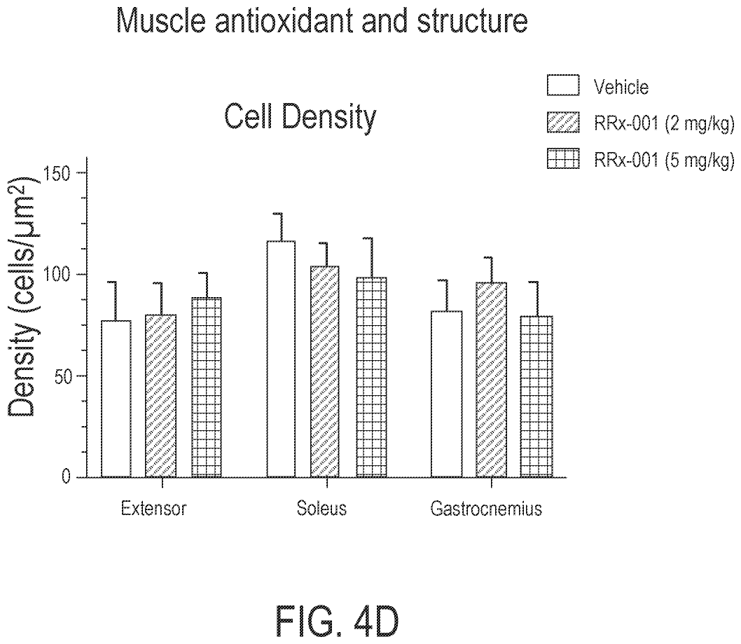

FIG. 4A shows a comparison of concentrations of malondialdehyde (MDA) in gastrocnemius, soleus, and extensor digitorum longus muscles in the mice. The exhaustive exercise-induced lipid peroxidation was estimated from a measurement of muscle levels of the MDA. As shown in FIG. 4A, the muscle MDA levels of the RRx-001-treated groups were significantly lower than those of the vehicle treated group (p<0.05).

FIG. 4B shows a comparison of concentrations of superoxide dismutase (SOD) in gastrocnemius, soleus, and extensor digitorum longus muscles. The data show that the mice in the RRx-001 group had higher concentrations of the SOD in the extensor and gastrocnemius muscles when compared to the vehicle group. The RRx-001 group that was administered a lower dose of RRx-001 (e.g., 2 mg/kg RRx-001) had a lower SOD concentration than both the vehicle group and the 5 mg/kg RRx-001 group in the soleus muscle while the 5 mg/kg RRx-001 group had the highest SOD concentration.

FIG. 4C shows a comparison of diameters of cells in the gastrocnemius, soleus, and extensor digitorum longus muscles after the 3-week study. The data shows that cells in the extensor and soleus muscles had a smaller diameter for the RRx-001 groups compared to the vehicle group at the end of the 3-week study. Cells in the gastrocnemius muscle had a larger diameter for the RRx-001 groups compared to the vehicle group.

FIG. 4D shows a comparison of densities of cells in the gastrocnemius, soleus, and extensor digitorum longus muscles. The data for cell densities of the extensor muscle shows that the RRx-001 groups (e.g., 2 mg/kg and 5 mg/kg RRx-001) had a higher cell density than the vehicle group where the RRx-001 group that was administered 5 mg/kg of the RRx-001 had a cell density that was higher than the other groups.

The data for cell densities of the soleus muscle shows that the RRx-001 groups had lower cell density than the vehicle group. The results for cell density in the gastrocnemius muscle showed that the vehicle group and 5 mg/kg RRx-001 group had a similar cell density, and the 2 mg/kg RRx-001 group had a higher cell density than the other groups.

These factors may all contribute to increased muscle conditioning, which would increase physical performance and reduce fatigue. The combinations of all factors may further result in an increase of motivation and adherence to exercise.

Many variations may be made to the embodiments described herein. All variations, including combinations of embodiments, are intended to be included within the scope of this disclosure. The description of the embodiments herein can be practiced in many ways. Any terminology used berein should not be construed as restricting the features or aspects of the disclosed subject matter. The scope should instead be construed in accordance with the appended claims.

What is claimed is:

1. A method for enhancing physical performance in a subject, the method comprising:
administering an effective amount of RRx-001, or a pharmaceutically acceptable salt thereof, to the subject in need thereof prior to the subject engaging in physical performance.

2. The method of claim 1, wherein the effective amount of RRx-001, or the pharmaceutically acceptable salt thereof, is a therapeutically effective amount of the RRx-001, or the pharmaceutically acceptable salt thereof.

3. The method of claim 2, wherein the therapeutically effective amount of the RRx-001, or the pharmaceutically acceptable salt thereof, is in a range of about 0.1 mg and about 500.0 mg.

4. The method of claim 3, wherein the therapeutically effective amount of the RRx-001, or the pharmaceutically acceptable salt thereof, is in a range of about 0.5 mg and about 200.0 mg.

5. The method of claim 3, wherein the therapeutically effective amount of the RRx-001, or the pharmaceutically acceptable salt thereof, is in a range of about 5 mg and about 50 mg.

6. The method of claim 3, wherein the therapeutically effective amount of the RRx-001, or the pharmaceutically acceptable salt thereof, is in a range of about 10 mg and about 30 mg.

7. The method of claim 1, wherein the subject is a mammal subject.

8. The method of claim 7, wherein the mammal subject is a human subject.

9. The method of claim 8, wherein the human subject is an athlete, a body builder, a manual worker, or an individual desiring to lose weight or enhance physique.

10. The method of claim 7, wherein the mammal subject is a non-human subject.

11. The method of claim 10, wherein the non-human subject is a livestock animal.

12. The method of claim 10, wherein the non-human subject is a domesticated animal.

13. The method of claim 10, wherein the non-human subject is a non-domesticated animal.

14. The method of claim 10, wherein the non-human subject is a horse.

15. The method of claim 10, wherein the non-human subject is a dog.

16. The method of claim 7, wherein the mammal subject is healthy.

17. The method of claim 7, wherein the mammal subject has or is suspected of having a condition selected from the group consisting of: hypercholesterolemia, hyperlipidemia, cancer, muscular dystrophies, peripheral vascular disease, patent foramen ovale, obesity, type 2 diabetes, angina pectoris, heart failure, mitochondrial disorders or diseases, chronic obstructive pulmonary disease (COPD), hyperCKemia, neurodegenerative disease, motor neuron disease, neuromuscular disease, multiple sclerosis, Charcot-Marie-Tooth disease, myositis including polymyositis and dermatomyositis, cardiovascular disease, pulmonary artery hypertension, insulin resistance, hypertension, myoedema, rhabdomyolysis, idiopathic chronic muscle fatigue, reduced skeletal muscle function, disrupted skeletal muscle function or metabolism, cardiac abnormalities, dysfunctional muscle, dysfunctional heart, and dysfunctional skeletal metabolism.

18. The method of any one of claims 1-17, wherein administering the effective amount of RRx-001, or the pharmaceutically acceptable salt thereof, occurs via oral administration, transdermal administration, administration by inhalation, nasal administration, topical administration, intra-aural administration, rectal administration, intravenous administration, intramuscular administration, subcutaneous administration, or intraperitoneal administration, or combinations thereof.

19. The method of any one of claims 1-17, wherein administering the effective amount of RRx-001, or the pharmaceutically acceptable salt thereof, occurs via parenteral administration.

20. The method of any one of claims 1-19, wherein administering the therapeutically effective amount of RRx-001, or the pharmaceutically acceptable salt thereof, is performed within twenty-four hours of the physical performance.

21. The method of any one of claims 1-20, wherein administering the therapeutically effective amount of RRx-001, or the pharmaceutically acceptable salt thereof, is performed via a single administration.

22. The method of any one of claims 1-20, wherein administering the therapeutically effective amount of RRx-001, or the pharmaceutically acceptable salt thereof, is performed via at least two administrations.

23. The method of claim 22, wherein administering the therapeutically effective amount of RRx-001, or the pharmaceutically acceptable salt thereof, is performed at a frequency of about once per day.

24. The method of claim 22, wherein administering the therapeutically effective amount of RRx-001, or the pharmaceutically acceptable salt thereof, is performed at a frequency of about once per week.

25. The method of claim 22, wherein administering the therapeutically effective amount of RRx-001, or the pharmaceutically acceptable salt thereof, is performed at a frequency of about twice per week.

26. The method of claim 22, wherein administering the therapeutically effective amount of RRx-001, or the pharmaceutically acceptable salt thereof, is performed at a frequency of about once per month.

27. The method of claim 22, wherein administering the therapeutically effective amount of RRx-001, or the pharmaceutically acceptable salt thereof, is performed at a frequency of about once every three months. 28 The method of any one of claims 1-27, wherein administering the therapeutically effective amount of RRx-001, or the pharmaceutically acceptable salt thereof, results in an increase in at least one of: VO₂ max, high-density lipoprotein (HDL) levels, or long-chain fatty acid (LCFA) muscle uptake.

29. The method of any one of claims 1-28, wherein administering the therapeutically effective amount of RRx-001, or the pharmaceutically acceptable salt thereof, results in an improvement in at least one of: left ventricular (LV) function, fasting glycemia, insulin resistance, fatty acid metabolism, fatty acid oxidation, or fatty acid utilization.

30. The method of any one of claims 1-29, wherein administering the therapeutically effective amount of RRx-001, or the pharmaceutically acceptable salt thereof, results in a decrease in at least one of: low-density lipoprotein (LDL), serum triglyceride (TAG) levels, blood pressure, slow skeletal troponin I concentration (sTnI), serum myoglobin concentration, scrum IL-1β, IL-6, decreased 8-hydroxy-2′-deoxyguanosine (8-OHdG), TNF-α concentration, or sedimentation rate and C-reactive protein.

31. The method of any one of claims 1-30, wherein administering the therapeutically effective amount of RRx-001, or the pharmaceutically acceptable salt thereof, results in a delayed onset of muscle soreness.

32. The method of any one of claims 1-31, further comprising:
administering an agent before, during, or after the administration of the effective amount of RRx-001, or the pharmaceutically acceptable salt thereof.

33. The method of claim 32, wherein the agent is selected from the group consisting of: an antioxidant, a vitamin, a mineral, a corticosteroid, a human growth hormone, a steroid, levothyroxine, erythropoietin, a selective androgen receptor modulator (SARM), human chorionic gonadotrophin (HCG), adrenocorticotrophin (ACTH), insulin, a beta 2 agonist, marijuana, a diuretic, a narcotic, a beta blocker, a nitrite, a nitrate, a nitric oxide donor, a PDE5inhibitor, a sympathomimetic drug, and an amino acid.

34. The method of claim 33, wherein the agent comprises the antioxidant, and wherein the antioxidant is selected from the group consisting of: lycopene, coenzyme Q10, melatonin, selenium, alpha-lipoic acid, ellagic acid, lutein, resveratrol, anthocyanins, ellagitannins, polyphenols, quercetin, and curcumin.

35. The method of claim 33, wherein the agent comprises the vitamin, and wherein the vitamin is selected from the group consisting of: Vitamin A, Vitamin C, Vitamin E, folic acid, and biopterin.

36. The method of claim 33, wherein the agent comprises the mineral, and wherein the mineral is selected from the group consisting of: calcium, phosphorus, potassium, sodium, chloride, magnesium, iron, zinc, iodine, chromium, copper, fluoride, molybdenum, and manganese.

37. The method of claim 33, wherein the agent comprises the nitric oxide donor, and wherein the nitric oxide donor comprises an organic nitrate.

38. The method of claim 37, wherein the organic nitrate is selected from the group consisting of: glyceryl trinitrate and isosorbide dinitrate.

39. The method of claim 33, wherein the agent comprises the nitric oxide donor, and wherein the nitric oxide donor comprises sodium nitroprusside.

40. The method of claim 33, wherein the agent comprises the nitric oxide donor, and wherein the nitric oxide donor comprises a sydnonimine.

41. The method of claim 40, wherein the sydnonimine is selected from the group consisting of: molsidomine and SIN-1.

42. The method of claim 33, wherein the agent comprises the nitric oxide donor, and wherein the nitric oxide donor comprises an S-nitrosothiol.

43. The method of claim 42, wherein the S-nitrosothiol is selected from the group consisting of: s-nitrosoglutathione and SNAP.

44. The method of claim 33, wherein the agent comprises the nitric oxide donor, and wherein the nitric oxide donor comprises a NONOate.

45. The method of claim 44, wherein the NONOate is selected from the group consisting of: spermine NONOate and DETA-NONOate.

46. The method of claim 32, wherein the agent comprises a PDE5 inhibitor.

47. The method of claim 46, wherein the PDE5 inhibitor is selected from the group consisting of: sildenafil, tadalafil, vardenafil, and avanafil.

48. The method of claim 32, wherein the agent comprises a sympathomimetic drug.

49. The method of claim 48, wherein the sympathomimetic drug is selected from the group consisting of dobutamine, albuterol, phenylpropanolamine, amphetamine, and ephedrine.

50. The method of claim 32, wherein the agent comprises an amino acid.

51. The method of claim 50, wherein the amino acid is selected from the group consisting of: L-camitine, L-creatine, L-taurine, arginine, and lysine.

52. The method of any one of claims 1-51, wherein the therapeutically effective amount of the RRx-001, or the pharmaceutically acceptable salt thereof, is administered as a composition comprising a blood product.

53. The method of claim 52, wherein the blood product comprises erythrocyte cells.

54. The method of claim 53, wherein the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution. and hypo-osmotic dialysis.

55. The method of claim 52, wherein the blood product is a mixture of packed red blood cells.

56. The method of claim 52, wherein the blood product is whole blood.

57. The method of claim 52, wherein the whole blood is autologous whole blood or donor-matched allogenic whole blood.

58. A composition for enhancing physical performance in a subject, the composition comprising an effective amount of RRx-001, or a pharmaceutically acceptable salt thereof, and at least one pharmaceutically acceptable carrier or excipient.

59. The composition of claim 58, wherein the effective amount of RRx-001, or the pharmaceutically acceptable salt thereof, is a therapeutically effective amount of the RRx-001, or the pharmaceutically acceptable salt thereof.

60. The composition of claim 59, wherein the therapeutically effective amount of the RRx-001, or the pharmaceutically acceptable salt thereof, is in a range of about 0.1 mg and about 500.0 mg.

61. The composition of claim 60, wherein the therapeutically effective amount of the RRx-001, or the pharmaceutically acceptable salt thereof, is in a range of about 0.5 mg and about 200.0 mg.

62. The composition of claim 60, wherein the therapeutically effective amount of the RRx-001, or the pharmaceutically acceptable salt thereof, is in a range of about 5 mg and about 50 mg.

63. The composition of claim 60, wherein the therapeutically effective amount of the RRx-001, or the pharmaceutically acceptable salt thereof, is in a range of about 10 mg and about 30 mg.

64. The composition of any one of claims 58-63, wherein the subject is a mammal subject.

65. The composition of claim 64, wherein the mammal subject is a human subject.

66. The composition of claim 64, wherein the mammal subject is a non-human subject.

67. The composition of claim 66, wherein the non-human subject is a livestock animal.

68. The composition of claim 66, wherein the non-human subject is a domesticated animal.

69. The composition of claim 66, wherein the non-human subject is a non-domesticated animal.

70. The composition of claim 64, wherein the mammal subject is healthy.

71. The composition of claim 64, wherein the mammal subject has or is suspected of having a condition selected from the group consisting of: hypercholesterolemia, hyperlipidemia, cancer, muscular dystrophies, peripheral vascular disease, patent foramen ovale, obesity, type 2 diabetes, angina pectoris, heart failure, mitochondrial disorders or diseases, chronic obstructive pulmonary disease (COPD), hyperCKemia, neurodegenerative disease, motor neuron disease, neuromuscular disease, multiple sclerosis, Charcot-Marie-Tooth disease, myositis including polymyositis and dermatomyositis, cardiovascular disease, pulmonary artery hypertension, insulin resistance, hypertension, myoedema, rhabdomyolysis, idiopathic chronic muscle fatigue, reduced skeletal muscle function, disrupted skeletal muscle function or metabolism, cardiac abnormalities, dysfunctional muscle, dysfunctional heart, and dysfunctional skeletal metabolism.

72. The composition of any one of claims 58-71, wherein the composition is administered via oral administration, transdermal administration, administration by inhalation, nasal administration, topical administration, intra-aural administration, rectal administration, intravenous administration, intramuscular administration, subcutaneous administration, or intraperitoneal administration, or combinations thereof.

73. The composition of claim 72, wherein the composition is administered via the oral administration, and wherein the composition is in a form of an oral rinse, a drink, an oral pill, or an oral tablet.

74. The composition of any one of claims 58-73, wherein the composition is administered within twenty-four hours of the physical performance.

75. The composition of any one of claims 58-74, wherein the composition is administered via a single administration.

76. The composition of any one of claims 58-74, wherein the composition is administered via at least two administrations.

77. The composition of claim 76, wherein the composition is administered at a frequency of about once per day.

78. The composition of claim 76, wherein the composition is administered at a frequency of about once per week.

79. The composition of claim 76, wherein the composition is administered at a frequency of about twice per week.

80. The composition of claim 76, wherein the composition is administered at a frequency of about once per month.

81. The composition of claim 76, wherein the composition is administered at a frequency of about once every three month.

82. The composition of any one of claims 58-81, wherein administration of the composition results in an increase in at least one of: VO₂ max, high-density lipoprotein (HDL) levels, or long-chain fatty acid (LCFA) muscle uptake.

83. The composition of any one of claims 58-82, wherein administration of the composition results in an improvement in at least one of: left ventricular (LV) function, fasting glycemia, insulin resistance, fatty acid metabolism, fatty acid oxidation, or fatty acid utilization.

84. The composition of any one of claims 58-83, wherein administration of the composition results in a decrease in at least one of: low-density lipoprotein (LDL), serum triglyceride (TAG) levels, blood pressure, slow skeletal troponin I concentration (sTnI), serum myoglobin concentration, serum IL-1β, IL-6, decreased 8-hydroxy-2′-deoxyguanosine (8-OHdG), TNF-α concentration, or sedimentation rate and C-reactive protein.

85. The composition of any one of claims 58-84, wherein administration of the composition results in a delayed onset of muscle soreness.

86. The composition of any one of claims 58-85, wherein the composition further comprises an agent.

87. The composition of claim 86, wherein the agent is selected from the group consisting of: an antioxidant, a vitamin, a mineral, a corticosteroid, a human growth hormone, a steroid, levothyroxine, erythropoietin, a selective androgen receptor modulator (SARM), human chorionic gonadotrophin (HCG), adrenocorticotrophin (ACTH), insulin, a beta 2 agonist, marijuana, a diuretic, a narcotic, a beta blocker, a nitrite, a nitrate, a nitric oxide donor, a PDE5 inhibitor, a sympathomimetic drug, and an amino acid.

88. The composition of claim 87, wherein the agent comprises the antioxidant, and wherein the antioxidant is selected from the group consisting of: lycopene, coenzyme Q10, melatonin, selenium, alpha-lipoic acid, ellagic acid, lutein, resveratrol, anthocyanins, ellagitannins, polyphenols, quercetin, and curcumin.

89. The composition of claim 87, wherein the agent comprises the vitamin, and wherein the vitamin is selected from the group consisting of: Vitamin A, Vitamin C, Vitamin E, folic acid, and biopterin.

90. The composition of claim 87, wherein the agent comprises the mineral, and wherein the mineral is selected from the group consisting of: calcium, phosphorus, potassium, sodium, chloride, magnesium, iron, zinc, iodine, chromium, copper, fluoride, molybdenum, and manganese.

91. The composition of claim 87, wherein the agent comprises the nitric oxide donor, and wherein the nitric oxide donor comprises an organic nitrate.

92. The composition of claim 91, wherein the organic nitrate is selected from the group consisting of: glyceryl trinitrate and isosorbide dinitrate.

93. The composition of claim 87, wherein the agent comprises the nitric oxide donor, and wherein the nitric oxide donor comprises sodium nitroprusside.

94. The composition of claim 87, wherein the agent comprises the nitric oxide donor, and wherein the nitric oxide donor comprises a sydnonimine.

95. The composition of claim 94, wherein the sydnonimine is selected from the group consisting of: molsidomine and SIN-1.

96. The composition of claim 87, wherein the agent comprises the nitric oxide donor, and wherein the nitric oxide donor comprises an S-nitrosothiol.

97. The composition of claim 96, wherein the S-nitrosothiol is selected from the group consisting of: s-nitrosoglutathione and SNAP.

98. The composition of claim 87, wherein the agent comprises the nitric oxide donor, and wherein the nitric oxide donor comprises a NONOate.

99. The composition of claim 98, wherein the NONOate is selected from the group consisting of: spermine NONOate and DETA-NONOate.

100. The composition of claim 86, wherein the agent comprises a PDE5 inhibitor.

101. The composition of claim 100, wherein the PDE5 inhibitor is selected from the group consisting of: sildenafil, tadalafil, vardenafil, and avanafil.

102. The composition of claim 86, wherein the agent comprises a sympathomimetic drug.

103. The composition of claim 102, wherein the sympathomimetic drug is selected from the group consisting of dobutamine, albuterol, phenylpropanolamine, amphetamine, and ephedrine.

104. The composition of claim 86, wherein the agent comprises an amino acid.

105. The composition of claim 104, wherein the amino acid is selected from the group consisting of: L-camitine, L-creatine, L-taurine, arginine, and lysine.

106. The composition of any one of claims 58-105, wherein the composition further comprises a blood product.

107. The composition of claim 106, wherein the blood product comprises erythrocyte cells.

108. The composition of claim 107, wherein the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis.

109. The composition of claim 106, wherein the blood product is a mixture of packed red blood cells.

110. The composition of claim 106, wherein the blood product is whole blood.

111. The composition of claim 106, wherein the whole blood is autologous whole blood or donor-matched allogenic whole blood.
